(19) United States
(12) Patent Application Publication
(10) Pub. No.: US 2024/0006058 A1
(43) Pub. Date: Jan. 4, 2024

(54) System and Method for Supporting an Integrated Healthcare Marketplace

(71) Applicant: Amos O. Dare, Boca Raton, FL (US)

(72) Inventor: Amos O. Dare, Boca Raton, FL (US)

(21) Appl. No.: 18/216,764

(22) Filed: Jun. 30, 2023

Related U.S. Application Data
(60) Provisional application No. 63/357,143, filed on Jun. 30, 2022.

(51) Int. Cl. G16H 40/20; G16H 50/20

(52) U.S. Cl. CPC G16H 40/20; G16H 50/20; G16H 10/60

(57) ABSTRACT

A system and method for electronically directing patient care includes a Marketplace Aggregator System. The marketplace aggregator 1) provides a portal for enrolling medical providers and patients, 2) provides a programming interface (API) for access to health applications (Health Application Marketplace), 3) integrates with third-party electronic health records (EHR) and health information networks (HINs) to create a unified patient FHIR database, 4) creates a health API to a unified patient FHIR database, 5) enables automated scheduling of appointments, 6) helps physicians compile a network of medical providers and medical services, including digital health and online services (Unique Provider Marketplace), 7) provides API for patient access to the Unique Provider Marketplace and the Health Application Marketplace, 8) helps patients create a Unique Patient Marketplace for receiving medical care, and 9) utilizes artificial intelligence and machine learning to predict optimal patient-provider matches based on training data from electronic health records.

### CROSS REFERENCE TO RELATED APPLICATION

This application claims the benefit of Provisional Application Ser. No. 63/357,143 filed by the present inventor on Jun. 30, 2022, the entire disclosure of which is incorporated herein by reference.

### BACKGROUND OF THE INVENTION

### Field of the Invention

The invention pertains to systems and methods for creating an integrated healthcare marketplace to help providers and their patients to electronically direct and receive medical care.

### Description of Related Art

In the current medical environment, the process of finding a readily available provider that accepts the patient's insurance in their geographic area can be frustrating and sometimes scary for patients and their treating providers. The problem is further compounded by the fact that no centralized electronic patient record systems yet exist. Medical providers are required to keep patient information in electronic health records (EHR) systems. Several EHR systems exist on the market for providers to use; unfortunately, these EHR systems lack interoperability which makes it difficult to transfer patient information from one provider EHR to another. This can block or hinder prompt evaluation, treatment, or transfer of care from one provider to another provider or for external services.

Consequently, doctors and their patients resort to outdated communication methods such as the fax, less efficient modalities such as telephone calls; and less secure channels such as email to communicate patient information among providers and their EHR. Although patients may have portal access to EHR system of their providers, most portals do not provide portability of the records, transfer of the records to the patient, or the ability of the patient to grant access to the records of one provider to another provider; a patient with multiple providers will therefore end up with multiple portals and no centralized medical records repository.

Decentralization of the patient's electronic record poses another problem for the patient when shopping for medical services or transferring care to another provider. It is nearly impossible to get all medical records transferred from all providers.

As a result of these difficulties, the provider-patient partnership suffers with the patient's treating provider becoming less involved in helping the patient find suitable providers and implementing care plans.

Finally, the abundance of digital health applications online lacks a sufficient platform to coordinate and guide their utilization by patients and their providers.

There is a pressing need for an integrated marketplace that brings together medical providers, ancillary medical providers, and digital healthcare applications, supported by a centralized patient EHR system. Such a system would enable the electronic direction of patient care, addressing the challenges faced in provider selection, information exchange, and care coordination. Additionally, there is a need for better algorithms and technologies to facilitate accurate patient-medical provider matching, optimizing the referral process and enhancing patient outcomes.

Objects and Advantages

Objects of the present invention include the following: 1. Providing a platform that centralizes patient electronic medical records, ensuring accessibility and availability of up-to-date patient information. 2. Facilitating seamless access to patient records by both patients and healthcare providers, enabling timely and informed decision-making in patient care. 3. Establishing a system for efficient transfer, exchange, and storage of patient records, including test results, consultation reports, vital signs, condition updates, and other relevant data. 4. Creating a health provider marketplace supported by a centralized patient record system. This marketplace assists providers and patients in collaboratively selecting and scheduling the most suitable available providers to implement the treating provider's plan of care. 5. Integrating health applications within the provider marketplace to enhance patient and provider utilization of these online services. The system provides support and guidance to ensure effective utilization of these applications. 6. Developing predictive algorithms based on artificial intelligence and machine learning, utilizing a training model as described in the Network Marketplace Aggregator System. These algorithms enable the system to predict optimal patient-medical provider matches, resulting in improved clinical outcomes and patient satisfaction.

### SUMMARY OF THE INVENTION

According to one aspect of the invention, a system for directing patient care electronically comprises:
a marketplace aggregator system that:
   provides a portal for enrolling medical providers, ancillary medical providers and patients;
   provides an application programming interface (API) for access to native and non-native health applications (Health Application Marketplace);
   integrates with third-party electronic health records (EHR) and health information networks (HINs);
   creates a health API to a hosted health record database;
   enables automated scheduling of medical appointments;
   allows a physician to compile a network of medical providers and medical services, including digital health and online services (Unique Provider Marketplace);
   provides an API for a patient to access the Unique Provider Marketplace and the Health Application Marketplace; and,
   helps the patient to create a Unique Patient Marketplace for receiving medical care.

According to another aspect of the invention, a system for directing patient care electronically comprises:
a server device hosting a marketplace aggregator system and a unified patient FHIR database that:
   provides a portal for enrolling medical providers, ancillary medical providers and patients;
   provides an application programming interface (API) for access to native and non-native health applications (Health Application Marketplace);
   integrates with third-party electronic health records (EHR) and health information networks (HINs);
   creates a health API to a hosted health record database;
   enables automated scheduling of medical appointments;
   allows a physician to compile a network of medical providers and medical services, including digital health and online services (Unique Provider Marketplace);
   provides an API for a patient to access the Unique Provider Marketplace and the Health Application Marketplace; and,
   helps the patient to create a Unique Patient Marketplace for receiving medical care; and,
a plurality of client devices comprising a user interface through which patients may access selected functions of the marketplace aggregator system.

According to another aspect of the invention, a method for directing patient care electronically comprises:
establishing a server device comprising a marketplace aggregator system and a unified patient FHIR database;
establishing a plurality of client devices each comprising a patient facing user interface;
providing a portal for enrolling medical providers, ancillary medical providers and patients;
accepting scheduling and referral information as inputs from users;
providing selected information as outputs to the client devices; and,
maintaining electronic health records in a format that is interoperable among selected EHR systems and for each patient contains a single integrated and unified patient record.

According to another aspect of the invention, a system for directing patient health care electronically comprises:
at least one memory device storing computer-executable instructions; and
at least one processor for executing the instructions stored on the at least one memory device, wherein execution of the instructions causes at least one processor to perform operations via a user interface of a Network Marketplace Aggregator System, said Network Marketplace Aggregator System comprising the totality of all entities, data, records, indexes, inputs, applications, APIs and services within the Provider Marketplace, Patient Marketplace, and Health Application Marketplace, wherein the operations comprise:
a) iteratively building a Network Marketplace comprising a plurality of database records associated with a respective plurality of Provider entities, Patient entities, and Healthcare Vendor entities, wherein building the database includes, for each entity of the plurality of entities, data fields that identify and characterize each entity for creating a Unique Provider Marketplace, a Unique Patient Marketplace, and a Health Application Marketplace;
b) maintaining a physician-selected subset of the Network Marketplace Aggregator System to create a Unique Provider Marketplace;
c) maintaining a patient-selected subset of the Network Marketplace Aggregator System to create a Unique Patient Marketplace;
d) helping the physician to compile his/her Unique Provider Marketplace of medical resources selected for use in management of patients by the physician, wherein the resources are selected from the group consisting of: primary/specialty/sub-specialty providers, ancillary medical services providers, laboratory testing facilities, radiology and imaging facilities, in-home and assisted living care, and online and remote monitoring services;
e) helping the patient to compile his/her Unique Patient Marketplace of medical providers, services, and applications actively engaged with and used by the Patient; and,
f) helping the Network Marketplace Aggregator System implementer in creating a Health Application Marketplace wherein native and non-native (third-party) applications can be developed and offered to patients at the patient's request or the request of a physician.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore non-limiting embodiments illustrated in the drawing figures, wherein like numerals (if they occur in more than one view) designate the same elements. The features in the drawings are not necessarily drawn to scale.

FIG. 1 is a schematic diagram of the overall system architecture of one example of the present invention.

FIG. 2 is a schematic diagram of an operating scenario in an example of the present invention.

FIG. 3 is a schematic diagram of an operating scenario in another example of the present invention.

FIG. 4 is a schematic diagram of an operating scenario in another example of the present invention.

FIG. 5 is a schematic diagram of an operating scenario in another example of the present invention.

FIG. 6 is a schematic diagram of the system architecture of a Marketplace Aggregator in accordance with some aspects of the present invention.

### DETAILED DESCRIPTION OF THE INVENTION

The invention provides a number of functions that are intended to improve the overall electronic patient management, typically initiated by a primary care provider (PCP) and electronically directing a patient to the services of one or more specialized health care providers. These specialized providers may be physicians (e.g., dermatologists, allergists, orthopedic surgeons, neurologists, psychiatrists, etc.), mental health professionals (e.g., psychologists), and other service providers (testing labs, home healthcare facilities, substance abuse facilities, etc.), as well as health online applications.

The invention further provides means for coordinating and scheduling appointments with specialized providers and integrating the EHR generated by such appointments into the patient's master EHR record so that the patient and his PCP may access the unified record data. Many additional functions and benefits may be provided, as will become clear in the examples that follow.

The basic elements of the inventive system include a server device and a plurality of patient facing client devices each having a user interface through which a patient can interact with the system in a number of ways, which will be described later.

The system may incorporate the use of artificial intelligence, specifically machine learning, to train a large language model. This language model is trained on data extracted from electronic health records (EHRs) to predict the best referral and clinical outcomes for matching patients to medical providers in the marketplace. By leveraging the power of machine learning, the system can analyze vast amounts of patient data, identify patterns, and make informed predictions about the most suitable healthcare providers for individual patients. This improves the efficiency and effectiveness of patient referrals, leading to better clinical outcomes and enhanced patient satisfaction.

The machine learning algorithms employed by the system learn from historical data, including patient demographics, medical conditions, treatment outcomes, and provider performance metrics. These algorithms can identify correlations and dependencies within the data to generate personalized recommendations for patients seeking medical care and deliver such recommendations to patients and medical providers via their respective user interfaces. By continuously learning and adapting from new data inputs, the system can refine its predictions and improve its accuracy over time, enhancing the overall patient-provider matching process.

The integration of artificial intelligence and machine learning within the marketplace aggregator system revolutionizes the way patient care is directed electronically. It enables a more precise and data-driven approach to match patients with the most suitable medical providers, enhancing the quality of care and optimizing healthcare resource allocation. Additionally, by leveraging electronic health records and the power of machine learning, the system facilitates seamless interoperability between different healthcare entities, improving the efficiency and effectiveness of patient management and fostering a comprehensive and integrated healthcare ecosystem.

Overall, the use of artificial intelligence and machine learning within the system enhances the patient experience, streamlines healthcare processes, and contributes to better health outcomes for individuals seeking medical care within the marketplace.

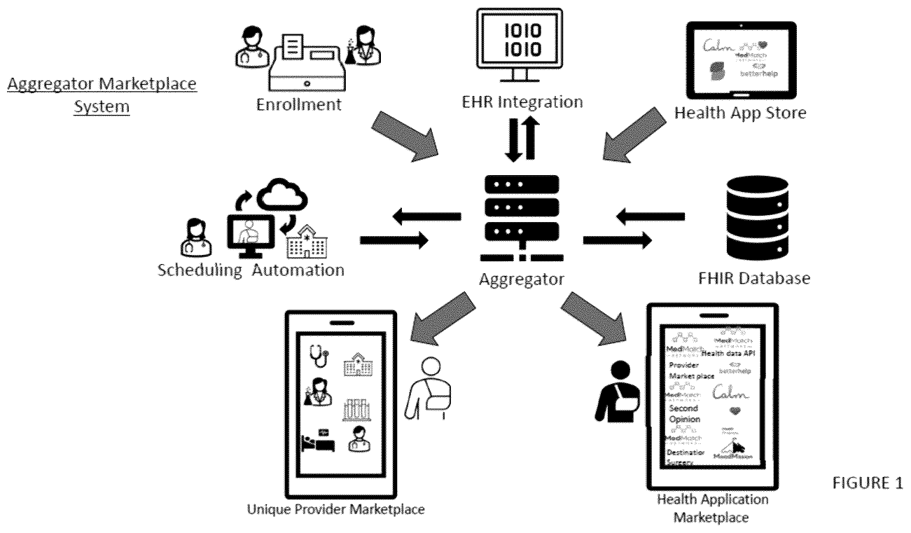

The server device includes a marketplace aggregator system and a unified patient FHIR database as shown generally in FIG. 1. Fast Healthcare Interoperability Resources (FHIR) is a standard describing data formats and elements (known as “resources”) and an application programming interface (API) for exchanging electronic health records. The standard was created by the Health Level Seven International (HL7) health-care standards organization. [See http://hl7.org/fhir/documentation.html for specification details, which are incorporated herein by reference in their entirety for background purposes.] Two patient-facing client devices are shown in FIG. 1; one client displays a Unique Provider Marketplace and the other client displays a Mobile Health Application Marketplace. Health applications (e.g., calm.com, betterhelp.com, MedMatch Network, etc.) are contained in a Health App Store as shown in the figure and may be downloaded to the client devices. Health care providers (upper left in FIG. 1), may enroll so that their information will become available for display in the Unique Provider Marketplace. The marketplace aggregator system interacts with automated scheduling systems to coordinate appointments, referrals, tests, and the like. The marketplace aggregator, using the unified patient FHIR database, integrates and updates EHR information as it is generated so that it may be accessed by patients and healthcare providers.

### Example

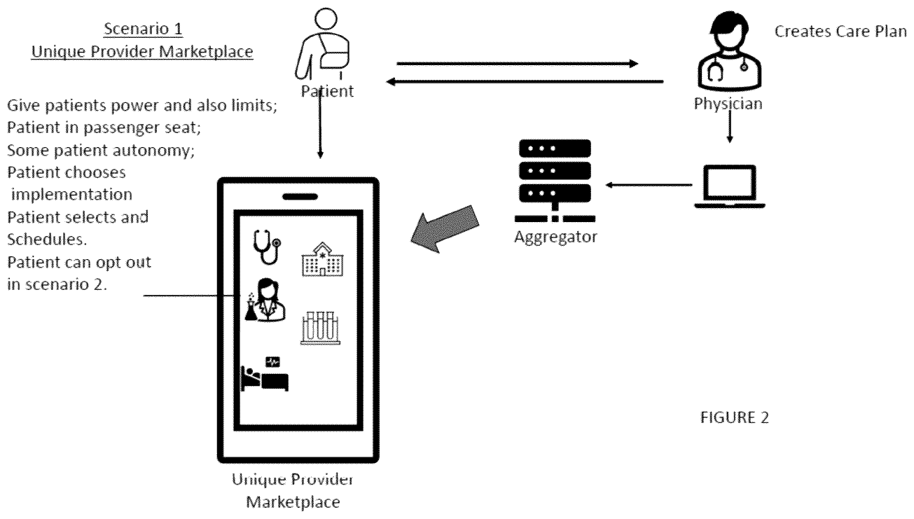

Use Case 1 (FIG. 2): Provider and patient use unique provider marketplace to manage the patient. The steps would typically include:
1. Patient sees doctor; doctor decides to send patient for medical services.
2. Doctor uses PC to log into Unique Provider Marketplace
3. Doctor sends patient a link (access code) to Unique Provider Marketplace
4. Patient accesses link provided by doctor to the Unique Provider Marketplace via Aggregator API
5. Patient enrolls via provided link
6. Patient receives marketplace options based on criteria specified by the doctor (diagnosis, location, healthcare network and cost)
7. Patient selects and schedules services
8. Aggregator automates and coordinates appointment scheduling
9. Patient receives services Providers upload report/test results to aggregator for doctor and patient to access.

### Example

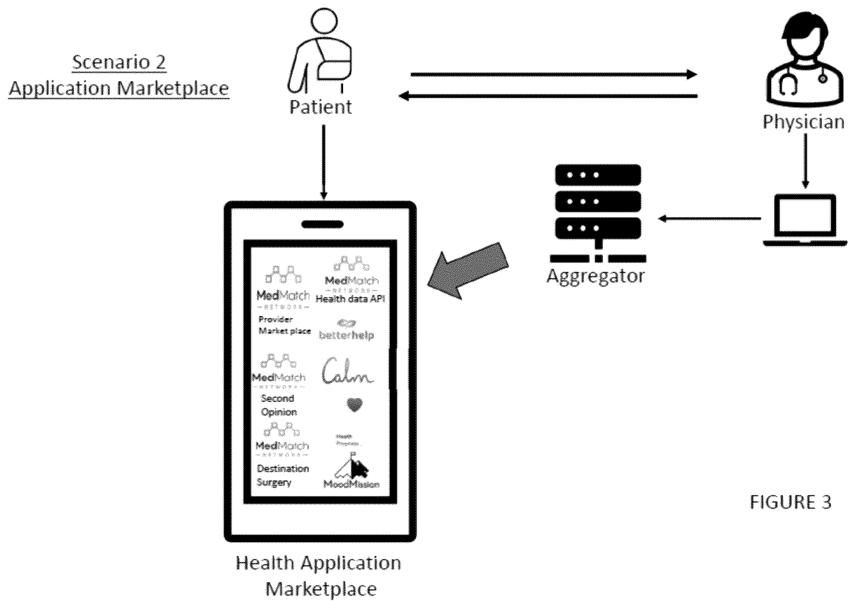

Use Case 2 (FIG. 3): Provider uses Unique Provider Marketplace and patient uses Health Application Marketplace to schedule services. The steps would typically include:
1. Patient sees doctor; doctor decides to send patient for medical services'
2. Doctor uses PC to log into Unique Provider Marketplace
3. Doctor sends patient a link (access code) to Unique Provider Marketplace
4. Patient accesses link provided by doctor to the Unique Provider Marketplace via Aggregator API Patient enrolls via provided link
6. Patient receives marketplace options based on criteria specified by the doctor (diagnosis, location, healthcare network and cost)
7. Patient accesses Health Application Marketplace via Aggregator API
8. Patient selects Health Application
9. Patient selects and schedules services Aggregator automates and coordinates appointment scheduling
11. Patient receives services
12. Providers upload report/test results to aggregator for doctor and patient to access.

### Example

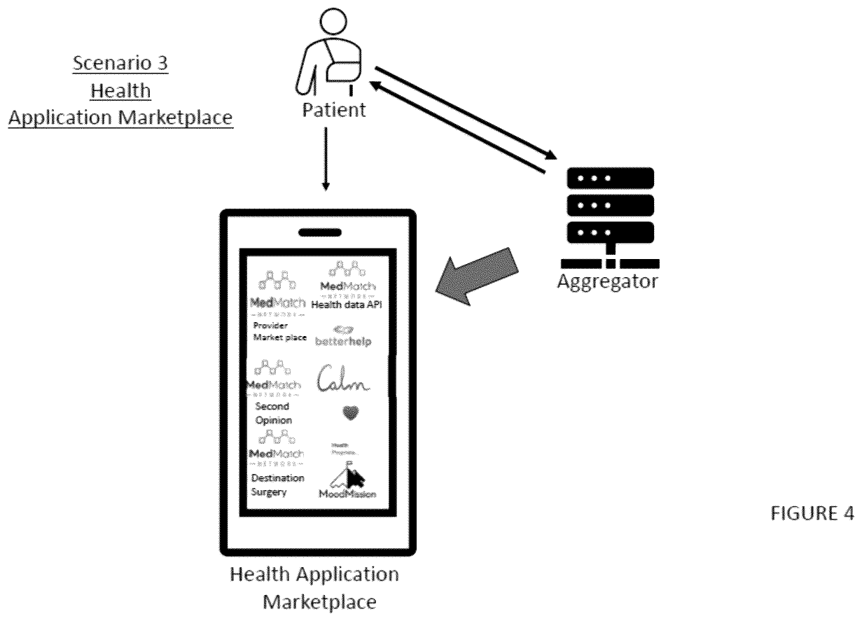

Use Case 3 (FIG. 4): Patient uses aggregator health application marketplace to schedule services (B2C Model). The steps would typically include:
1. Patient enrolls on Aggregator
2. Patient accesses Health Application Marketplace via Aggregator API
3. Patient selects Health Application
4. Patient selects and schedules services Aggregator automates and coordinates appointment scheduling
6. Patient receives services
7. Providers upload report/test results to aggregator for doctor and patient to access.

### Example

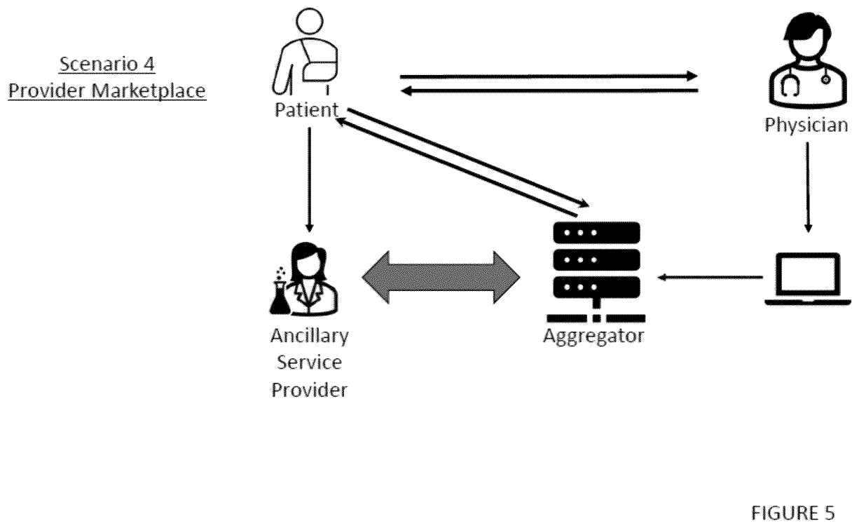

Use Case 4 (FIG. 5): Doctor uses Aggregator Unique Provider Marketplace to schedule services. The steps in this process have been implemented and include:
1. Doctor used PC to log into Unique Provider Marketplace
2. Doctor selected and scheduled services
3. Aggregator automated and coordinated appointment scheduling
4. Patient received services
5. Providers uploaded report/test results to aggregator for doctor and patient to access.

The Network Marketplace Aggregator System.

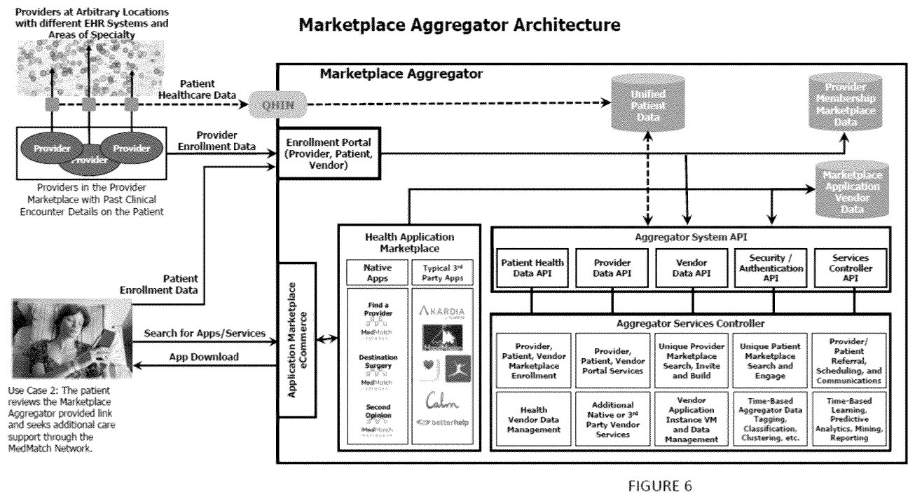

FIG. 6 presents an example of the Network Marketplace Aggregator architecture. The system comprises:
at least one memory storing computer-executable instructions; and at least one processor for executing the instructions stored on at least one memory, wherein execution of the instructions causes at least one processor to perform operations for enhancing a user interface of a Network Marketplace Aggregator System, the operations comprising:
a) iteratively building a Network Marketplace comprising a plurality of database records associated with a respective plurality of Provider entities, Patient entities, and Healthcare Vendor entities, wherein building the database includes, for each entity of the plurality of entities, data fields necessary for creating a Unique Provider Marketplace, a Unique Patient Marketplace, and a Health Application Marketplace;
b) the totality of all entities, data, records, indexes, inputs, applications, APIs and services within the Provider Marketplace, Patient Marketplace, and Health Application Marketplace forms the Network Marketplace Aggregator System. Physician-selected entities constitute the Unique Provider Marketplace, while patient-selected entities constitute the Unique Patient Marketplace;
c) helping physicians to compile a Unique Provider Marketplace by selecting and connecting with other Network Providers and Ancillary Service Providers. This includes primary/specialty/sub-specialty providers, ancillary medical services providers, and online and remote monitoring services.
d) helping patients to compile a Unique Patient Marketplace, encompassing medical providers, services, and actively used applications.
e) facilitating the creation of a Health Application Marketplace, where native and non-native applications are developed and offered to patients based on their requests or physicians' recommendations.

The system may enable a Physician to receive an invitation to enroll in the Network Marketplace Aggregator System as an approved Network Provider. Once enrolled, the Marketplace Aggregator System will help the Provider to:
a) create a Network Provider profile consisting of professional credentials, licensing information areas of expertise or specialization; services offered, insurance participation, office hours, etc.;
b) create a Unique Provider Marketplace by selecting, inviting and establishing connection with other Network Providers and Ancillary Service Providers; and,
c) direct patient care electronically.

The system may also allow an Ancillary Service Provider to receive an invitation to enroll in the Network Marketplace Aggregator System as an approved Network Provider; once enrolled, the Marketplace Aggregator System will help the Provider to:
a) create a Network Provider profile consisting of professional credentials, licensing information areas of expertise or specialization; services offered, insurance participation, office hours, etc., and,
b) create a Unique Provider Marketplace by selecting, inviting and establishing connection with other Network Providers and Ancillary Service Providers.

The system may enable a patient to use his or her provider's Unique Provider Marketplace to review Marketplace Providers and schedule visits. Decision-supporting information may be presented to the patient for selection of Marketplace Providers including metrics captured from previous referring providers and their patients. Selection criteria for Marketplace Providers may include professional credentials, areas of expertise, and performance ratings, provider schedule and availability, and other data supporting the patient's decision process for selecting a Provider on the Healthcare Marketplace.

The system may enable a Patient to access the Health Application Marketplace to review and receive digital health services or Marketplace providers to select alternatives to those entities included in the eMatch; decision-supporting information is presented to the patient for selection of Marketplace Providers including metrics captured from previous referring providers and their patients. Healthcare Applications Selection criteria for Healthcare Applications may include information pages provided by the Healthcare Applications vendors, including at least product features, license terms, and costs.

The system may enable a Physician to use his or her Unique Provider Marketplace to electronically direct patient care (eMatch). This might include communicating to the patient a link for the patient to enroll and log into the Network Marketplace Aggregator System. The communication may be done via a patient preferred electronic communications modality, including but not limited to SMS, email, healthcare portal account message, or mobile device application notification.

The system may enable a Physician to utilize his Unique Provider Marketplace to select patient-relevant entities from within his Unique Provider Marketplace, choose a specific Marketplace Provider, and automatically schedule an appointment with the selected Marketplace Provider on behalf of the patient. The Network Marketplace Aggregator System would then automate the confirmations, rescheduling, reminders, and making test results and reports available.

The system may allow the Physician to create his own Unique Provider Marketplace using a Provider Marketplace search function and/or a Health Application Marketplace search function to identify and select entities for directing patient care. Provider Marketplace search available to enrolled Providers may include filter criteria including but not limited to Marketplace Provider license credentials, professional credentials, areas of expertise, and performance ratings. Physician-selected Provider Marketplace entities may be electronically messaged with an invitation to join the physician's Unique Provider Marketplace. Invited Provider Marketplace entities may receive a link to login to the Network Marketplace Aggregator System if the Physician or Service Provider is enrolled, or to create a new account if the Physician or Service Provider is not enrolled.

The Network Marketplace Aggregator System preferably employs open system standards and provides an application programming interface access to the General Network Marketplace Aggregator System, including the Provider Marketplace, the Patient Marketplace, and the Health Application Marketplace; the Network Marketplace Aggregator System API consists of a collection of system-specific API libraries, including, but not limited to, a Patient Health Data API, a Provider Marketplace API, a Patient Marketplace API, a Health Application Marketplace Vendor API. All Network Marketplace Aggregator System APIs can be used specifically on entities within the Unique Provider Marketplace and the Unique Patient Marketplace.

The Network Marketplace Aggregator System enables automatic segmentation, instance classification, and the keyword tagging of provider, patient, encounter, and vendor data sets for use as input into learning systems for recognition of patient engagement, provider engagement, service utilization, and health application utilization, among others.

The Network Marketplace Aggregator System enables a plurality of classification, recognition, learning, data mining, predictive analytics, analysis, and reporting workflows or processes on the provider, patient, and encounter data sets.

The system may further comprise a Health Application Marketplace, the operations comprising:
a programming interface for integration of new applications that extend the default features of the Healthcare Application Marketplace or add new customer application features;
enabling applications that natively integrate with and have secure access to Provider Marketplace entity, Patient Marketplace entity, Healthcare Application Marketplace Vendor data from independently designed applications on a single interface;
creating an applications catalog, configurable and searchable based on features and functionality created by the application vendor;
enabling Provider or Patient browsing of available Health Application Marketplace entities based on segmentation and keywords defined by the MedMatch Network Marketplace Aggregator System implementor;
enabling the end-user to purchase of Health Applications for which the Health Application vendor has placed a usage cost on the application; and,
enabling the end-user to create an account, with secure credential-based access, for display and tracking of purchases, communications with Health Application Marketplace vendors, technical support tickets, etc.

For each patent enrolled in the Network Marketplace Aggregator System, the system may:
create and maintain a unified individual Patient Health Data Record, comprising current and historical health data, information, reports, and findings obtained through patient encounters within the Provider Marketplace;
integrate with third-party electronic health record systems (EHRs) and health information networks (HINs) to receive Patient Health Data for aggregation into the Network Marketplace Aggregator System creating or updating unified patient records;
present the physician with existing aggregated medical records, from all available sources; and,
secure queries to create, read, update, and delete entire Healthcare Marketplace Provider Master Records or Patient Master Integrated Health Data Records, or specific fields within one or more respective records.

The Network Marketplace Aggregator System preferably encompasses a comprehensive set of features and AI-driven capabilities to streamline patient-provider matching, facilitate healthcare applications, and optimize electronic patient care coordination.

1. A system for directing patient care electronically comprising:
a server device hosting a marketplace aggregator system and a unified patient Fast Healthcare Interoperability Resources (FHIR) database, wherein said marketplace aggregator system comprises:
a portal for enrolling medical providers, ancillary medical providers and patients;
a health application marketplace comprising an application programming interface (API) giving users access to native and non-native health applications; and,
wherein said server device communicates with third-party electronic health records (EHR) and health information networks (HINs) and is interoperable therewith; and,
a plurality of client devices comprising a user interface through which medical providers and patients access selected functions of said marketplace aggregator system.

2. The system of claim 1 wherein said server comprises a health application programming interface (health API) to a health record database hosted on said server.

3. The system of claim 1 wherein said server is interoperable with third-party scheduling systems so that said portal can be used by said medical providers, ancillary medical providers, and patients for automated scheduling of appointments.

4. The system of claim 1 wherein said marketplace aggregator system comprises a unique provider marketplace in which a medical provider can use a client device to compile a network of medical providers and medical services including digital health and online medical services.

5. The system of claim 4 wherein said marketplace aggregator system comprises an application programming interface through which a patient can access said unique provider marketplace and said health application marketplace.

6. The system of claim 1 wherein said marketplace aggregator system includes a user-created unique patient marketplace through which individual patients may receive medical care.

7. The system of claim 1 wherein said marketplace aggregator system:
applies artificial intelligence and machine learning algorithms to data in one or more databases to generate personalized recommendations for matching patients and medical providers; and,
delivers said personalized recommendations to users via their respective client devices.

8. The system of claim 7 wherein said data is selected from the group consisting of: historical patient data, demographic data, medical conditions, treatment outcomes, and provider performance metrics.

9. A method for electronically directing patient care comprising:
providing a server device comprising a marketplace aggregator system and a unified patient FHIR database, wherein said marketplace aggregator system comprises:
a portal for enrolling medical providers, ancillary medical providers and patients; and,
a health application marketplace comprising an application programming interface (API) giving users access to native and non-native health applications;
providing a data connection by which said server communicates with selected third-party electronic health records and health information networks;
providing a plurality of client devices each comprising at least one of: a patient facing user interface, and a medical provider facing user interface; and,
delivering health care recommendations to patients and medical providers via said user interface(s).

10. The method of claim 9 wherein said server comprises a health application programming interface (health API) to a health record database hosted on said server.

11. The method of claim 9 wherein said server is interoperable with third-party scheduling systems so that said portal can be used by said medical providers, ancillary medical providers, and patients for automated scheduling of appointments.

12. The method of claim 9 wherein said marketplace aggregator system comprises a unique provider marketplace in which a medical provider can use a client device to compile a network of medical providers and medical services including digital health and online medical services.

13. The method of claim 12 wherein said marketplace aggregator system comprises an application programming interface through which a patient can access said unique provider marketplace and said health application marketplace.

14. The method of claim 9 wherein said marketplace aggregator system includes a user-created unique patient marketplace through which individual patients may receive medical care.

15. The method of claim 9 wherein said marketplace aggregator system:
applies artificial intelligence and machine learning algorithms to data in one or more databases to generate personalized recommendations for matching patients and medical providers; and,
delivers said personalized recommendations to users via their respective client devices.

16. The method of claim 7 wherein said data is selected from the group consisting of:
historical patient data, demographic data, medical conditions, treatment outcomes, and provider performance metrics.

17. A system for directing patient health care electronically comprising:
at least one memory device storing computer-executable instructions; and
at least one processor for executing said instructions stored on said at least one memory device, wherein execution of said instructions causes at least one processor to perform operations via a user interface of a network marketplace aggregator system, said network marketplace aggregator system comprising the entities, data, records, indexes, inputs, applications, APIs and services within a provider marketplace, a patient marketplace, and health application marketplace, wherein said operations comprise:
a) iteratively building a Network Marketplace comprising a plurality of database records associated with a respective plurality of provider entities, patient entities, and healthcare vendor entities, wherein building the database includes, for each entity of said plurality of entities, data fields that identify and characterize said entity for creating a unique provider marketplace, a unique patient marketplace, and a health application marketplace;
b) maintaining a physician-selected subset of said network marketplace aggregator system to create a unique provider marketplace associated with said physician;
c) maintaining a patient-selected subset of the network marketplace aggregator system to create a unique patient marketplace associated with said patient;
d) helping said physician to compile his/her unique provider marketplace of medical resources selected for use in management of patients by said physician, wherein said resources are selected from the group consisting of: primary care providers, /specialty care providers, sub-specialty care providers, ancillary medical services providers, laboratory testing facilities, radiology and imaging facilities, in-home and assisted living care, and online and remote monitoring services;
e) helping said patient to compile his/her unique patient marketplace of medical providers, services, and applications actively engaged with and used by said patient; and,
f) helping the network marketplace aggregator system implementer in creating a health application marketplace wherein native applications and non-native (third-party) applications can be developed and offered to patients at said patients' request or at the request of a physician.

18. The system of claim 17 wherein said operations further comprise:
g) accepting scheduling and referral information as inputs from users.

19. The system of claim 17 wherein said operations further comprise:
h) maintaining electronic health records in a format that is interoperable among selected EHR systems and for each patient contains a single integrated and unified patient record.

20. The system of claim 17 wherein said operations further comprise:
i) receiving selected information as inputs from client devices and providing selected information as outputs to said client devices.
